(19) United States
(12) Patent Application Publication
(10) Pub. No.: US 2022/0241156 A1
(43) Pub. Date: Aug. 4, 2022

(54) CHILD-RESISTANT DISPENSERS FOR THIN STRIP MEDICATIONS

(71) Applicant: Sunovion Pharmaceuticals Inc., Marlborough, MA (US)

(72) Inventors: Thierry BILBAULT, Cambridge, MA (US); Arthur TONG, Mendon, MA (US)

(73) Assignee: Sunovion Pharmaceuticals Inc., Marlborough, MA (US)

(21) Appl. No.: 17/622,046

(22) PCT Filed: Jun. 26, 2020
(86) PCT No.: PCT/US2020/039874

§ 371 Date: Dec. 22, 2021

Related U.S. Application Data
(60) Provisional application No. 62/867,414, filed on Jun. 27, 2019.

(51) Int. Cl. A61J 7/00; A61K 9/70; A61J 1/00; B65D 50/00

(52) U.S. Cl. CPC A61J 7/0076; A61K 9/7007; B65D 2215/00; B65D 50/00; A61J 1/00

(57) ABSTRACT

Product containers, especially child-resistant product dispensers, and methods of use thereof. In some particular embodiments, provided herein are child-resistant medication dispensers, and methods of use thereof for administering medications to individuals that may suffer from manual dexterity problems. The disclosure is particularly useful in dispensing orally dissolving medications intended for sublingual application, such as orally dissolving strips. In one embodiment of the disclosure a medication dispenser is disclosed comprising a container having an opening and an inner chamber, a cap configured to close the opening of the container and provide a child-resistant locking engagement with the container, and a tray deployable in the chamber of the container and configured to hold at least one pouch carrying a dose of medication. The tray can include a support configured to hold one or more pouches when the tray and pouches are deployed within the container.

### TECHNICAL FIELD

Provided herein are product containers, including but not limited to child-resistant product dispensers, and methods of use thereof. In some particular embodiments, provided herein are child-resistant medication dispensers, and methods of use thereof for administering medications to individuals that may suffer from manual dexterity problems.

### BACKGROUND

Since about 1973, U.S. federal regulations promulgated under the Poison Prevention Packaging Act have required that prescription drugs intended for oral administration to humans comply with certain “child-resistant” packaging requirements unless specifically exempted. Generally speaking, child-resistant packaging is specially designed or constructed to be difficult for children under 5 years of age to open. Ideally, such child-resistant packaging is also designed so as not to present undue difficulties for patients accessing their medication. However, child-resistant packaging can sometimes pose a challenge for adults, particularly elderly or infirm individuals or those with impaired motor skills, e.g., those afflicted with neurological or muscular disorders.

Moreover, certain illnesses can make common pharmaceutical delivery vehicles, such as pills, capsules or tablets difficult for individuals to ingest. For example, many illnesses such as Parkinson's Disease, Duchenne Muscular Dystrophy and Amyotrophic Lateral Sclerosis (ALS) are accompanied by difficulties in swallowing (dysphagia).

Unfortunately, some individuals that are prescribed medications to treat a disease or disorder (e.g., Parkinson's Disease or related symptoms) also suffer from poor manual dexterity (e.g., hand tremors, decreased coordination between hands or loss of fine motor control skills) and find it difficult to open child-resistant drug containers, generally. These difficulties can be compounded when the medication is in the form of a thin strip medication. Thus, there is a need for a child-resistant device, and methods of use thereof, for administering medications to individuals that may suffer from manual dexterity problems.

### SUMMARY

Devices are disclosed comprising a container having an opening and an inner chamber, a cap configured to close the opening of the container, and a tray deployable in the chamber of the container and configured to hold at least one pouch carrying a product.

In some embodiments, the device is a medication dispenser, the cap is a child-resistant cap, and the tray deployable in the chamber of the container is configured to hold at least one pouch carrying a dose of medication. In some embodiments, the tray comprises a support configured to hold one or more pouches when the tray and pouches are deployed within the container, and optionally wherein the support comprises a rail onto which the pouches can be individually coupled to facilitate dispensation of one pouch at a time by pulling the pouch from the rail. In some embodiments, each of the pouches comprises an opening for attachment to the rail. In some embodiments, the openings of the pouches are larger than the thickness of the rail such that the pouches can extend partially out of the opening of the container when the container is uncapped and inverted.

In some embodiments, the tray is foldable to facilitate insertion of the tray into the chamber of the container. In some embodiments, the tray comprises at least two arms and a hinge about which the arms can fold. In some embodiments, at least one arm of the tray further comprises a spar that forms a rail to support the pouch when the tray is folded, and optionally wherein the spar couples to the second arm when the tray is folded. In some embodiments, the tray further comprises a first spar and a second spar that engage with each other to form the rail when the tray is deployed within the chamber of the container. In some embodiments, one of the spars has a male end and the other has a female end that can be joined together to form the rail, and optionally wherein the first and second spars are configured to snap-fasten together to form the rail. In some embodiments, each arm further comprises a flange configured to engage an inner rim of the container to impede removal of the tray after insertion into the chamber of the container. In some embodiments, the flange of at least one arm is shaped to extend circumferentially along the container rim such that it subtends an angle of at least 10 degrees. In some embodiments, the flange of each arm is shaped to extend circumferentially along the container rim such that it subtends an angle of at least 30 degrees.

In some embodiments, the container further comprises at least one post that protrudes inwardly into the chamber of the container to engage the tray when deployed therein, and the tray comprises an opening for receiving the post. In some embodiments, the tray opening is a longitudinal slot that permits limited longitudinal movement of the tray within the chamber such that the tray and associated pouches can extend partially out of the opening of the container when the container is uncapped and inverted. In some embodiments, the cap and container cooperate to provide a child resistant locking engagement. In some embodiments, the engagement of the cap and container can be unlocked by a quarter turn action, or push and twist action by a squeeze and twist action.

In some embodiments, the device further comprises a plurality of pouches. In some embodiments, each pouch contains a dose of a thin strip medication. In some embodiments, the thin strip medication is an orally-dissolvable medication.

In some embodiments, the inner chamber comprises at least one (e.g., 1, 2, 3, or 4) inner protrusion. In some embodiments, the at least one inner protrusion limits or prevents rotation of the dispensing tray inside the inner chamber. In some embodiments, the inner chamber comprises one inner protrusion. In some embodiments, the inner chamber comprises two inner protrusions. In some embodiments, the inner chamber comprises four inner protrusions. In some embodiments, the cap comprises a plurality of vertical ribs disposed along the outer circumference of the cap. In some embodiments, the cap comprises a mouth having one or more threads disposed along its circumference.

Also disclosed are methods of dispensing a product comprising the steps of: providing at least one sealed pouch containing a product, folding a tray around the pouch such that the tray supports the pouch, placing the tray and supported pouch inside a container such that the tray is engaged by the container in a manner that impedes removal of the tray, and securing the container with a cap configured to close the opening of the container, whereby a user can remove the cap and obtain an individual pouch by reaching into the container and pulling the pouch from its supporting tray.

In some embodiments, the method further comprises a method of dispensing a medication, the pouch contains a dose of medication and the cap is a child-resistant cap.

In some embodiments, the tray comprises a support configured to hold a plurality of pouches when the tray and pouches are deployed within the container, and optionally the tray comprises a rail onto which the pouches can be individually coupled to facilitate dispensation of one pouch at a time by pulling the pouch from the rail. In some embodiments, each of the pouches comprises an opening for attachment to the rail. In some embodiments, the openings of the pouches are larger than the thickness of the rail such that the pouches can extend partially out of the opening of the container when the container is uncapped and inverted. In some embodiments, the tray is foldable along a hinge region to facilitate insertion of the tray into the chamber of the container. In some embodiments, the tray comprises at least two arms and a hinge about which the arms can fold, and the step of placing the tray inside the container further comprises folding the arms about the hinge region and placing the hinge region in proximity to a bottom surface of the container. In some embodiments, at least one arm of the tray further comprises a spar and the step of folding the tray further comprises forming a rail to support the pouch when the tray is folded, and optionally wherein the spar couples to the second arm when the tray is folded. In some embodiments, the tray further comprises a first spar on a first arm and a second spar on a second arm that engage with each other to form the rail when the tray is deployed within the chamber of the container, and the step of folding the tray further comprises forming the rail such that it passes through a hole in the pouch. In some embodiments, one of the spars has a male end and the other has a female end that can be joined together to form the rail, and optionally wherein the first and second spars are configured to snap-fasten together to form the rail. In some embodiments, each arm further comprises a flange configured to engage an inner rim of the container to impede removal of the tray after insertion into the chamber of the container. In some embodiments, the flange of at least one arm is shaped to extend circumferentially along the container rim such that it subtends an angle of at least 10 degrees. In some embodiments, the flange of each arm is shaped to extend circumferentially along the container rim such that it subtends an angle of at least 30 degrees.

In some embodiments, the container further comprises at least one post that protrudes inwardly into the chamber of the container to engage at least one opening in the tray when deployed therein, and the step of placing the tray inside the container further comprises placing the tray such that the tray opening receives the post.

In some embodiments, the tray opening is a longitudinal slot that permits limited longitudinal movement of the tray within the chamber such that the tray and associated pouches can extend partially out of the opening of the container when the container is uncapped and inverted.

In some embodiments, the engagement of the cap and container can be unlocked by a push and twist action. In some embodiments, the engagement of the cap and container can be unlocked by a squeeze and twist action.

In some embodiments, the method further comprises supporting a plurality of pouches with the tray inside the container. In some embodiments, each pouch contains a dose of thin strip medication. In some embodiments, the thin strip medication is orally-dissolvable medication.

In some embodiments, the inner chamber comprises at least one (e.g., 1, 2, 3, or 4) inner protrusion. In some embodiments, the inner chamber comprises one inner protrusion. In some embodiments, the inner chamber comprises two inner protrusions. In some embodiments, the inner chamber comprises four inner protrusions. In some embodiments, the at least one inner protrusion limits or prevents rotation of the dispensing tray inside the inner chamber. In some embodiments, the cap comprises a plurality of vertical ribs disposed along the outer circumference of the cap. In some embodiments, the cap comprises a mouth having one or more threads disposed along its circumference.

In one aspect, methods of treating a subject afflicted with a neurological condition are disclosed comprising the steps of: providing at least one sealed pouch containing a medication, folding a tray around the pouch such that the tray supports the pouch, placing the tray and supported pouch inside a container such that the tray is engaged by the container in a manner that impedes removal of the tray, and securing the container with a cap configured to close the opening of the container, thereby allowing the subject to remove the cap and obtain an individual pouch by reaching into the container and pulling the pouch from its supporting tray and further permitting the subject to open the pouch and administer to themselves the medication.

In some embodiments, the cap is a child-resistant cap and the method further comprises releasing the interlocking engagement of the cap and container. In some embodiments, the neurological condition is Parkinson's Disease. In some embodiments, the neurological condition is an off episode associated with Parkinson's Disease. In some embodiments, the medication comprises a thin strip medication deployed within the pouch. In some embodiments, the method further comprises opening the pouch by pulling on opposing tabs of the pouch to separate the pouch into at least two sheet elements and allow the subject to access the thin strip medication deployed between the sheet elements. In some embodiments, the medication comprises apomorphine or a pharmaceutically acceptable salt thereof. In some embodiments, the apomorphine or a pharmaceutically acceptable salt thereof is formulated for sublingual release and the method permits the subject to place the thin strip medications under their tongue.

### BRIEF DESCRIPTION OF THE DRAWINGS

In order that the disclosure may be well understood, there will now be described various forms thereof, given by way of example, reference being made to the accompanying drawings, with like numbers used in the figures to refer to like components, elements, portions, regions, openings, apertures, etc., in which:

FIG. 1 is a side view of a container according to the disclosure with a product dispensing tray deployed therein;

FIG. 2 is a plan view of one embodiment of a dispenser tray prior to deployment within a dispenser;

FIG. 3 is a schematic perspective view of a dispenser according to the disclosure;

FIG. 4A is a plan view of a medication pouch for use in a dispenser according to the disclosure;

FIG. 4B is a side view of a partially opened medication pouch of FIG. 4A;

FIG. 4C is schematic perspective view of the partially opened pouch of FIG. 4A;

FIG. 5 is side view of the dispenser of FIG. 1 with a plurality of pouches coupled to a dispenser rail;

FIG. 6 is another side view of the dispenser of FIG. 5, showing partial extension of the pouches past the dispenser mouth when the dispenser is inverted;

FIG. 7 is a side view of an alternative embodiment of a dispenser tray according to the disclosure;

FIG. 8 is a side view of a dispenser according to the disclosure with the tray of FIG. 7 deployed therein.

FIG. 9 is a side view of another alternative embodiment of a dispenser tray according to the disclosure employing a single rail for support of pouches;

FIG. 10 is a side view of a dispenser according to the disclosure with the tray of FIG. 9 deployed therein;

FIG. 11 is perspective view of yet another embodiment of a device according to the disclosure having “thumb notches” along the container mouth to further assist a user in removing pouches;

FIG. 12 is a perspective view of another embodiment of a dispensing tray according to the disclosure; the dispensing tray includes struts, which provide additional structural rigidity;

FIG. 13 is a plan view of the dispensing tray shown in FIG. 12;

FIGS. 14 and 15 are side views of the dispensing tray shown in FIG. 12;

FIG. 16 is a perspective view of another embodiment of a container according to the disclosure; the container is configured to accommodate dispensing trays disclosed herein with or without pouches;

FIGS. 17 and 20 are side views of the container shown in FIG. 16;

FIG. 18 is a top view of the container shown in FIG. 16;

FIG. 19 is a bottom view of the container shown in FIG. 16;

FIG. 21 is a vertical, cross-section view of the container shown in FIG. 16;

FIG. 22 is a horizontal, cross-section view of the container shown in FIG. 16;

FIG. 23 is a perspective view of another embodiment of a container according to the disclosure; the container is configured to accommodate dispensing trays disclosed herein with or without pouches;

FIG. 24 is a side view of the container shown in FIG. 23;

FIG. 25 is a horizontal, cross-section view of the container shown in FIG. 23;

FIGS. 26 and 27 are side views of another embodiment of a dispensing ray according to the disclosure; the dispensing tray is shown in its folded configuration;

FIG. 28 is a perspective view of the dispensing tray shown in FIG. 26;

FIG. 29 is a plan view of a medication pouch for use in a dispenser according to the disclosure;

FIG. 30 is a schematic perspective view of a dispenser according to the disclosure;

FIGS. 31 and 32 are side views of the dispenser shown in FIG. 30;

FIG. 33 is a horizontal, cross-section view of the dispenser shown in FIG. 30; and

FIG. 34 is a vertical, cross-section view of the dispenser shown in FIG. 30.

### DETAILED DESCRIPTION

Described herein are product containers, including but not limited to child-resistant product dispensers, and methods of use thereof, for providing products, such as medications, to individuals that may suffer from manual dexterity problems. Embodiments disclosed herein may be particularly useful in dispensing thin strip medications, such as orally dissolving medications intended for sublingual application.

In some embodiments, a device is provided, e.g., a product dispenser, comprising a container having an opening and an inner chamber, a cap configured to close the opening (e.g., mouth) of the container and provide a child-resistant locking engagement with the container, and a tray deployable in the chamber of the container and configured to hold at least one pouch carrying a dose of medication. The tray can comprise a support configured to hold one or more pouches when the tray and pouches are deployed within the container.

For example, the support can be a rail onto which the pouches can be individually coupled to facilitate dispensation of one pouch at a time by pulling the pouch from the rail. Each of the pouches can comprise an opening for attachment to the rail. In one embodiment, the tray can have a first spar and a second spar on opposing wings of the tray that engage with each other to form the rail when the tray is folded about a hinge portion and deployed within the chamber of the container. By way of further example, one of the spars can have a male end and the other can have a female end such that that can be joined together to form the rail. Alternatively, or in addition, the spars can be joined together by a snap-fasten connection. In another embodiment, a single spar can be used to form the rail and the tray can be designed such that, upon folding, the spar spans a substantial portion of the container width. The single spar can extend from one wing of the tray and connect with a slot, snap-fasten or other joinder mechanism on the other wing of the tray when the tray is folded about a hinge portion.

In certain embodiments, the openings of the pouches can be larger than the thickness of the rail such that the pouches can extend partially out of the opening of the container when the container is uncapped and inverted.

Preferably, the tray is foldable to facilitate its insertion into the chamber of the container, e.g., the tray can comprise at least two arms and a hinge about which the arms can fold. Each arm can comprise an end portion or flange configured to engage an inner rim of the container to impede removal of the tray after insertion into the chamber of the container. The end portion can be curved or otherwise shaped to extend circumferentially along the container rim such that it subtends an angle of at least 10 degrees, or at least 20 degrees, or at least 30 degrees or at least 40 degrees.

In certain embodiments, the container can comprise at least one post that protrudes inwardly into the chamber of the container to engage the tray when deployed therein, and the tray comprises an opening for receiving the post. The tray opening can be a longitudinal slot that permits limited longitudinal movement of the tray within the chamber such that the tray and associated pouches can extend partially out of the opening of the container when the container is uncapped and inverted.

The engagement of the cap and container can be unlocked by a “quarter-turn” action, “push and twist” action or by a “squeeze and twist” action or any other of numerous well-known similar child-resistant engagement mechanisms.

The devices of the present disclosure are preferably capable of supporting a plurality of pouches with each pouch containing a single dose of thin strip medication, e.g., a thin strip orally-dissolvable medication.

In other embodiments of the disclosure, methods of dispensing medication are disclosed that can comprise the steps of providing at least one sealed pouch containing a dose of medication, folding a tray around the pouch such it supports the pouch, placing the tray and supported pouch inside a container such that the tray is engaged by the container in a manner that impedes removal of the tray, and securing the container with a cap configured to close the opening of the container and provide a child-resistant locking engagement with the container, whereby an adult user of the device can remove the cap and obtain an individual pouch by reaching into the container and pulling the pouch from its supporting tray.

In certain embodiments of the inventive methods, the tray comprises a support configured to hold a plurality of pouches when the tray and pouches are deployed within the container. The support can be a rail onto which the pouches can be individually coupled to facilitate dispensation of one pouch at a time by pulling the pouch from the rail. Each of the pouches can comprise an opening for attachment to the rail. Preferably, the openings of the pouches are larger than the thickness of the rail such that the pouches can extend partially out of the opening of the container when the container is uncapped and inverted.

The tray can comprise a first spar and a second spar that engage with each other to form the rail when the tray is deployed within the chamber of the container, and the step of folding the tray can further comprise forming the rail such that it passes through a hole in the pouch. By way of example, one of the spars can have a male end and the other can have a female end. Folding the tray causes the first and second spars to join together to form the rail. The first and second spars can also be configured to snap-fasten together to form the rail.

The methods of the disclosure can be practiced with a tray that is foldable along a hinge region to facilitate insertion of the tray into the chamber of the container. For example, the tray can comprise at least two arms and a hinge about which the arms can fold, and the step of placing the tray inside the container further comprises placing the hinge region along a bottom surface of the container. Each arm further can have an end portion or flange configured to engage an inner rim of the container to impede removal of the tray after insertion into the chamber of the container. The end portion of one or both arms can also be shaped to extend circumferentially along the container rim such that it subtends an angle of at least 10 degrees, or at least 20 degrees, or at least 30 degrees, or at least 40 degrees.

The methods can also be practiced with a container having at least one post that protrudes inwardly into the chamber of the container to engage at least one opening in the tray. The step of placing the tray inside the container further comprises placing the tray such that the tray opening receives the post. In certain embodiments, the tray opening can be a longitudinal slot that permits limited longitudinal movement of the tray within the chamber such that the tray and associated pouches can extend partially out of the opening of the container when the container is uncapped and inverted.

In certain embodiments, the container mouth can further comprise one or more “Thumb-notches” to allow the user more accessibility. In certain preferred embodiments, two thumb-notches opposite of one another are provided in the container mouth. The thumb-notches can be of equal size or one notch smaller than the other. The thumb-notches can give the end-user more access to grabbing and removing a pouch. In these embodiments it can further be advantageous for the cap to have two downward extending flaps (or one flap in the case of a single thumb-notch configuration) to provide closure of the mouth when the cap and container are in the closed or “locked” position.

The methods disclosed herein are preferably practiced to support a plurality of pouches with the tray inside the container where each pouch contains a dose of thin strip medication, e.g., a thin strip orally dissolvable medication.

It should be noted that, as used in this specification and the appended claims, the singular forms “a,” “an,” and “the” include plural referents unless the content clearly dictates otherwise. Thus, for example, reference to a composition containing “a support” includes two or more supports. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or arranged to perform a particular task or adopt a particular configuration. The phrase “configured” can be used interchangeably with other similar phrases such as arranged and configured, constructed and arranged, constructed, manufactured and arranged, adapted, and the like.

The term “child-resistant,” as used herein with reference to a cap or the combination of a cap and container, is intended to broadly encompass closure mechanisms designed or constructed to be difficult for children under 5 years of age to open. For example, the engagement of the cap and container can be unlocked by a “push and twist” action, a “quarter turn action,” or a “squeeze and twist” action, or by any one of numerous other, well-known, child-resistant closures.

The term “spar” as used herein is intended to encompass any element that provides support for a product. Preferably, the spar serves as a rail either by itself or in conjunction with another spar. For example, the spar can be a protrusion, post, bar, pole, or beam formed on, or attached to, at least one arm of a tray.

The term “snap-fasten” as used herein is intended to encompass any one of numerous interlocking fastener mechanisms for joining structures (e.g., spars) together. For example, in the case of a first spar having a male end and a second spar having a female end, a annular ridge or groove can be formed on the inside of the female spar and a mating annular groove or ridge is formed on the outside of the male spar. More generally, a snap fastener can take the form of any protrusion that mates with a recess. In some embodiments, snap-fastening can be achieved by simply relying on frictional engagement of two structures.

The term “thin strip” in the context of medications is intended to encompass drug delivery vehicles in the form of strips, films, membranes, foils, patches, or sheets. They are shaped such that one dimension, e.g., the thickness or z-dimension, is substantially thinner than at least one other dimension, e.g., the width or length (the x-dimension or y-dimension), or both other dimensions. The thin strip has width and length dimensions that are significantly larger than its thickness dimension. For example, the width and length can each be at least ten times the thickness dimension, or preferably at least one-hundred times the thickness dimension. Typically, the z-dimension is about 0.01 to about 100 micrometers thick, while the surface area defined by the width and length, e.g. the x-y surface, of the thin strip ranges from about 1 to about 20 cm². Thin strip medications can be orally dissolving medications but are not limited thereto.

Thin strips (e.g., orally dissolving thin strips) can be advantageous for such individuals because the drug can be applied under the tongue (sublingually) and need not be swallowed.

The terms “neurological condition,” or “neurological disorder,” or “neurological disease” are used interchangeably herein, unless indicated otherwise, and intended to encompass pathologies of the brain, spinal cord, nerves or neuromuscular systems, generally, including but not limited to Parkinson's Disease, ALS, Alzheimer's disease, Bell's palsy, muscular dystrophy, multiple sclerosis and epilepsy.

In certain embodiments, the devices and methods of the disclosure can be used to assist subjects afflicted such neurological conditions. For example, Parkinson's disease is characterized by a lack of dopamine, a signaling molecule in the brain, which can cause afflicted individuals to experience tremors, stiffness, and slowness. A typical approach for treating these symptoms is to administer a dopamine precursor (e.g., levodopa) that can cross the blood-brain barrier more readily than dopamine. Inside the brain, the dopamine precursor is converted into dopamine, thereby easing motor problems.

However, with long-term use the beneficial effects of levodopa or similar dopamine precursor can wear off before a new dose can be taken. The period when the medication has a positive effect on Parkinson's symptoms is called “on-time.” Once the medication stops working, a so-called “off-episode” occurs where symptoms recur. Off-episodes are particularly problematic with prolonged use of levodopa because the body's ability to convert levodopa into dopamine decreases, lowering the medication's efficacy.

The term “off-episode” or variations thereof (e.g., “off condition”) is intended to encompass end-of-dose wearing “off” (including early morning “off”), partial/delayed/No-ON and unpredictable “off” situations where the subject is not responding to a Parkinson's medication).

Apomorphine is an agent known as being useful in the treatment of “OFF” episodes associated with Parkinson's disease. Sunovion Pharmaceuticals has recently developed a sublingual (under the tongue) formulation of apomorphine (APL-130277). Sublingual administration is particularly useful because Parkinson's patients often also exhibit difficulties in swallowing pills.

Thus, the present disclosures can provide devices and methods to assist subjects afflicted with neurological conditions, such as Parkinson's disease, who wish to take apomorphine during their off-episodes. The present disclosure can provide child-resistant devices and methods for administering apomorphine and other medicines to Parkinson's subject who may also have limited fine motor skills.

One method of treating a subject afflicted with a neurological condition, such as Parkinson's disease, can comprise the steps of providing at least one sealed pouch containing a medication, folding a tray around the pouch such it supports the pouch, placing the tray and supported pouch inside a container such that the tray is engaged by the container in a manner that impedes removal of the tray, securing the container with a cap configured to close the opening of the container, allowing the subject to remove the cap and obtain an individual pouch by reaching into the container and pulling the pouch from its supporting tray and further permitting the subject to open the pouch and administer to themselves the medication.

The method can further be useful when the cap is a child-resistant cap and the method further comprises releasing the interlocking engagement of the cap and container. The method can be especially useful when the subject is experiencing an off-episode associated with Parkinson's disease.

The medication can comprise a thin strip medication within the pouch, which can be opened by pulling on opposing tabs of the pouch to separate the pouch into at least sheet elements and allow the subject to access the thin strip medication deployed between the sheet elements. In certain embodiments, the medication can comprise apomorphine, especially apomorphine that is formulated for sublingual release, such that the subject can place the thin strip apomorphine-containing medication under their tongue.

In certain embodiments, the subject may wish to drink and swallow water to help the medication dissolve. Once an individual pouch is removed from the container, a wing tab (described in more detail below) can be held in each hand between the thumb and finger. The wing tabs can be gently pulled apart to open the pouch. Then the sublingual thin strip medication (e.g., a film) can be extracted from the pouch. The film can be placed close to the base of the tongue. The subject should try to avoid chewing, or swallowing the film or saliva. Typically, the film will dissolve in about 2 minutes.

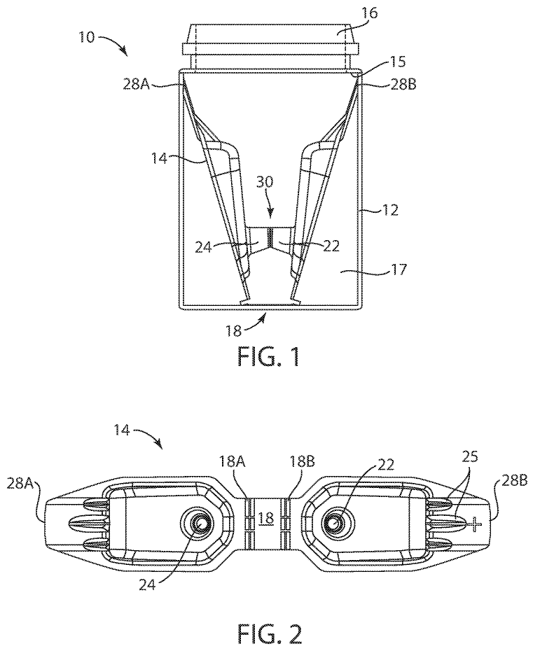

FIG. 1 illustrates a device 10 according to the disclosure comprising a container 12 having an open mouth 13 and an inner chamber 17. Deployed within the chamber is a dispensing tray 14. The tray 14 is preferably a foldable tray comprising two arms 28A, 28B configured to fold along a hinge region 18 for insertion through the mouth of the container 12. Once folded and inserted, distal ends of the arms 28A and 28B preferably engage the container 12, e.g., an inner rim 15 of the container, to block removal. In the illustrated embodiment, the tray 14 further comprises a first spar 22 and a second spar 24 that contact each other to form a support 30 when the tray is in its folded condition. As illustrated, the spars 22, 24 form a rail extending laterally across at least a portion of the inner chamber of the container to secure one or more pouches (not shown), each pouch carrying a dose of medication.

The device 10 further comprises a cap 16 to provide closure of the container 12. The cap 16 and container 12 preferably form a child-resistant closure, e.g., a “quarter-turn” releasable engagement, a “push down and turn” releasable engagement or a “squeeze and turn” releasable engagement. In a “quarter-turn” closure, the cap must be twisted approximately a quarter turn before it can be lifted off. In a “push down and turn” child-resistant closure, the cap is biased such that a down force must be applied to engage threads on outer rim of the container to unscrew the cap from the container. Typically, only a quarter turn is necessary to separate the cap from the container. In “squeeze and turn” closures, the sides of the cap can be pushed in, typically to create clearance between pegs inside the cap and pegs on the outside of the container to be able to twist it open. Although “push down and turn” and “squeeze and turn” caps are often preferable because of their simplicity and common acceptance, it should be clear that any one of a wide variety of child-resistant locking mechanisms can be employed to provide closure of the container. For further descriptions of various child-resistant caps, see “Standard Classification of Child-Resistant Packages,” ASTM Standard D3475-18, (available from the ASTM website) herein incorporated by reference in its entirety.

FIG. 2 is a plan view of a dispensing tray 14 according to the disclosure in its pre-folded configuration. The tray comprises hinge region 18 and arms 28A, 28B. Struts 25 provide additional structural rigidity to inhibit deformation except along the edges 18A and 18B of hinge region 18. Distal end portions of the arm 28A and 28B can be curved to further conform to the inner rim 15 around the mouth 13 of container 12. Spars 22 and 24 can be adapted to engage one another when folded, e.g., via a male-female coupling or via any one of a variety of mating or snap-fastening type fittings.

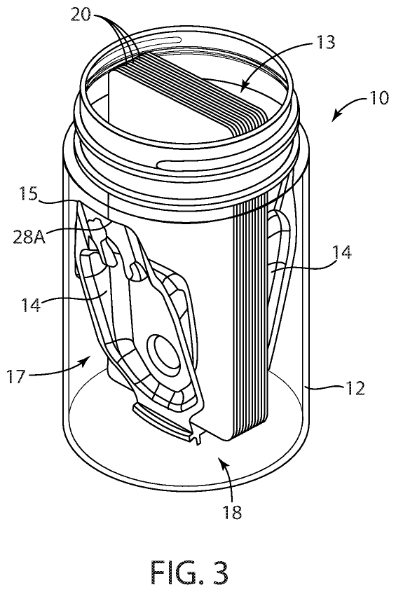

FIG. 3 is a schematic perspective view of the container 12 of the device 10 with tray 14 folded (at the hinge region 18). The folded tray 14 has been passed through container mouth 13 and placed within the container 12 such that the trays' end portions 28A and 28B engage the inner rim 15. Also shown is a plurality of medication pouches 20 attached to the support 30 (snared by the rail formed by spars 22 and 24).

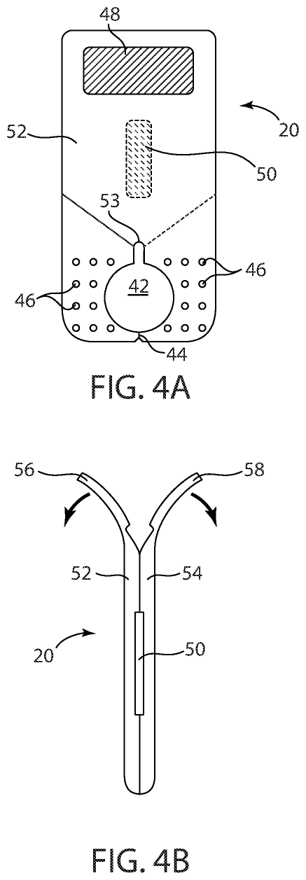

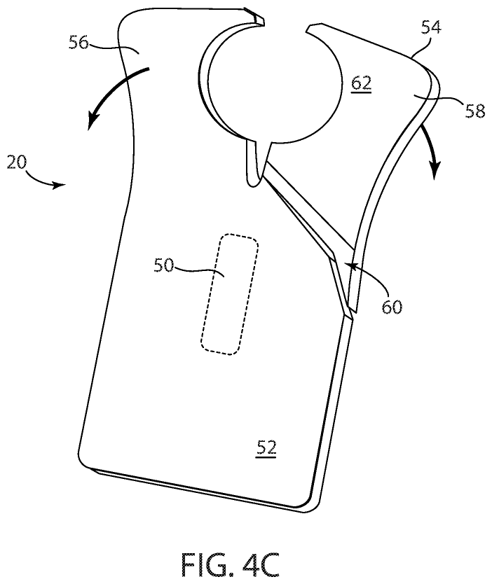

FIGS. 4A and 29 are plan views of a medication pouch 20 for use with the devices 10, 110, and 120 of the disclosure. Pouch 20 is designed to encapsulate a thin strip medication 50 (shown in phantom). Pouch 20 can comprise a front side sheet 52 and a back-side sheet (not shown in this view) as well as mounting hole 42 for engagement with the support element of the dispenser tray. For example, with reference as well to FIGS. 1-3, a plurality of pouches can be loaded onto spar 22 or spar 24 (or both) before the tray is folded. Once folded, the spars form a rail to hold the pouches in place within the container 12. Pouch mounting hole 42 is preferably larger than the rail to permit some degree of vertical movement of the pouches and facilitate easy extraction of an individual pouch. In addition to the large mounting hole 42, the pouch can also comprise a small slit (or a perforated “tear” line) 44 to further facilitate dispensing of a pouch when it is pulled out of the container. Tactile or textured features, e.g., dimples, 46 can be placed on one side, or the other, of each pouch (or on both sides) to maintain a spaced apart relationship between individual pouches and help ensure only a single pouch is detached at one time. The tactile features can also assist in opening the pouch, as described in more detail below. A grip 48 on another portion of the pouch 20 (e.g., a textured region of surface 52) can further assist in extraction.

FIG. 4B is cross-section side view of a partially opened pouch 20. In the exemplary construction, pouch 20 is formed joining a first sheet 52 and a second sheet 54 together with a thin strip medication 50 encapsulated between the two sheets. Preferably, the sheets are sealed in such a manner that they can be separated. For example, sheets 52 and 54 can be bound together at least partially by a low shear strength adhesive. For example, tabs 56 and 58 can take the form of “rabbit ears” and the medication 50 can be accessed by pulling tabs 56 and 58 in opposite directions to separate the main portions of sheets 52 and 54 from each other.

FIG. 4C is a schematic perspective view of a pouch 20, further illustrating a process for obtaining an encapsulated thin strip medication from the pouch. By pulling tab 56 in one direction and pulling tab 58 in a laterally opposite direction, an opening 60 between the sheets is formed. The pouch can be further configured (e.g., via the use of a different higher strength adhesive) such that an upper portion 62 of sheet 52 is pulled away with back side sheet 54 and preferably a similar split in sheet 54 occurs on the other side of the pouch (not shown).

Generally speaking, pouches for use in the present disclosure can comprise a first sheet layer and a second sheet layer. The first sheet layer can be overlaid on top of the second sheet layer. The first layer can be coextensive with the second layer so that the first layer and the second layer share a common outer perimeter. Some portions of the first layer can be sealed or joined with corresponding portions of the second layer to define one or more pouch seal zones. Some portions of the first layer can remain separate, unsealed, or not joined with the second layer to define one or more unsealed zones. The pouch seal zone can enclose an unsealed pouch zone in which a thin strip medication can be placed. The pouch seal zone thus protects the thin strip medication from exposure.

The pouch can further comprise one or more tabs in a header zone. In some examples, the first sheet layer and the second sheet layer can be sealed together at a first area to create a first tab, and sealed together at a second area to create a second tab. The first tab and the second tab can be separated from the pouch seal zone by an unsealed portion of the pouch. The unsealed portion of the pouch can extend from a first edge of the pouch to a second edge of the pouch opposite the first edge. The first tab and the second tab can be separated by an edge of the pouch that defines a void between the first tab and the second tab.

In some examples, the pouch comprises a first tab-cut on a first side of the pouch (for example, the front of the pouch) and a second tab-cut on the opposite side of the pouch (for example, the back of the pouch). The first tab-cut is a cut through the first layer of the pouch that does not reach the second layer of the pouch. The second tab-cut is a cut through the second layer of the pouch that does not reach the first layer. In some examples the first tab-cut and the second tab-cut do not overlap.

In some examples, the first tab-cut begins at a first edge of the pouch and ends at an edge of the pouch defining the void between the first tab and the second tab. In some examples, the second tab-cut begins at a second edge of the pouch opposite the first edge of the pouch, and ends at the edge of the pouch defining the void between the first tab and the second tab. In some examples, the first tab-cut does not intersect the second tab-cut. In some examples, the first tab-cut and the second tab-cut are both located in the unsealed portion of the pouch. In some examples, the first tab-cut is parallel to a first edge of the pouch seal zone and the second tab-cut is parallel to a second edge of the pouch seal zone. In some examples, the first tab-cut is not parallel to the second tab-cut.

As noted above, the first and second tabs can comprise one or more tactile features. The tactile features can be physical features located on a tab that allow the pouch to be easier to open, such as by decreasing the likelihood that a user's fingers will slip or slide on the tab, or by helping the user to identify the location of the tabs and the best location for grasping the pouch during opening. For example, in lieu of the illustrated dimples, the tactile feature can be an aperture, one or more slits, grooves, recesses or a region of increased surface roughness.

Pouch 20 functions by allowing a user to access the enclosed thin strip medication by gripping the first and second tabs, 56 and 58, and then pulling the tabs in opposite directions. The seal is broken when the first sheet layer is peeled apart from the second sheet layer. Pulling the tabs apart causes the first layer to separate from the second layer by breaking the seal between the first layer and the second layer in the pouch seal zone.

In some embodiments, the pouch seal zone has an apex 53 in the shape of a chevron, as shown in FIG. 4A. Pulling the first and the second tabs apart can cause the seal to be broken first at the apex of the pouch seal zone. Wide seal areas can require more force to break the seal than narrow seal areas. At the apex, the width of the seal is narrow. The narrow chevron shape requires relatively little force to overcome the seal. The force needed to peel the first layer apart from the second layer increases as the seal gets wider across with width of the pouch at the portion of the pouch seal zone adjacent to the unsealed area of the header zone. Eventually, pulling the first and second tabs farther and farther apart causes the side edge portions of the pouch seal zone to become unsealed, which exposes the thin strip medication contained within the unsealed area of the pouch.

Further details and alternative constructions for pouches that may be used in the present disclosure can be found in WO2017/214428 and US2017/0355504, the disclosures of which are incorporated herein by reference in their entirety.

The thin strip medication can take a variety of forms, depending, for example, on the nature of the medication and the release mechanism. Orally dissolving thin strip medications, for example, can have a soluble film layer, a coating applied on at least one side of the film layer, and an active ingredient that is contained in the coating, the film layer, or both the coating and film layer. The film layer may be soluble in water and/or saliva and/or other aqueous solutions. In other embodiments, the film layer can be hydrophobic.

The thin strip can take any shape that is suitable for oral administration or applicable to mucous membranes of various body parts of a subject. The film strip may be generally rectangular in shape. In one embodiment, the film strip may be generally rectangular in shape having a dimension of about ½ inch by 1 inch. Alternatively, the film strip may be circular with a diameter of about ⅜ inch. In certain embodiments, the thin strip can have a thickness of about 0.01 to 5 mm. In other embodiments, the film layer can have a thickness of about 0.03 to 2 mm or about 0.04 to 1 mm. Such a film strip may be suitable for application to mucous membranes located in other areas of the body besides the mouth.

The thin strip medication, can be constructed to have an acceptable dissolution rate in the oral cavity or on various mucous membranes of a subject. The composition may dissolve in about 1 second to 5 minutes, preferably, in about 1 second to 3 minutes, or more rapidly in about 10 seconds to 60 seconds.

The film layer may be made from a natural or synthetic polymer. In certain embodiments, the polymer is water soluble. In other embodiments, the polymer may be soluble in saliva and/or other aqueous solutions and/or the polymer may be hydrophobic. Preferably, the polymer has good film moldability, produces a soft flexible film and is safe for human consumption. The polymer may be a water-soluble cellulose derivative such as hydroxypropyl cellulose (HPC), methyl cellulose, hydroxypropyl alkylcellulose, carboxymethyl cellulose or the salt of carboxymethyl cellulose. Alternatively, the polymer may comprise an acrylic acid copolymer or its sodium, potassium or ammonium salt. The acrylic acid copolymer or its salt may be combined with methacrylic acid, styrene or vinyl type of ether as a comonomer, poly vinyl alcohol, poly vinyl pyrrolidone, polyalkylene glycol, hydroxy propyl starch, alginic acid or its salt, poly-saccharide or its derivatives such as trangacanth, bum gelatin, collagen, denatured gelatin, and collagen treated with succinic acid or anhydrous phthalic acid. The film layer may be made from pullulan, maltodextrin, pectin, alginates, carrageenan, guar gum, or other gelatins. The film layer can optionally contain additives known in the art.

Non-limiting examples of thin strips can be found, e.g., in WO2009/052421 the disclosure of which is incorporated herein by reference in their entirety.

Further non-limiting examples of thin strips that can be dispensed according to the disclosure include sublingual apomorphine films for the treatment of Parkinson's disease. Such films and other sublingual thin strips may be found in WO2010/144817, WO2012/083269, WO2016/172095, US2012/0195995, US2011/0111011, and US2018/0133146, the disclosures of which are incorporated herein by reference in their entirety.

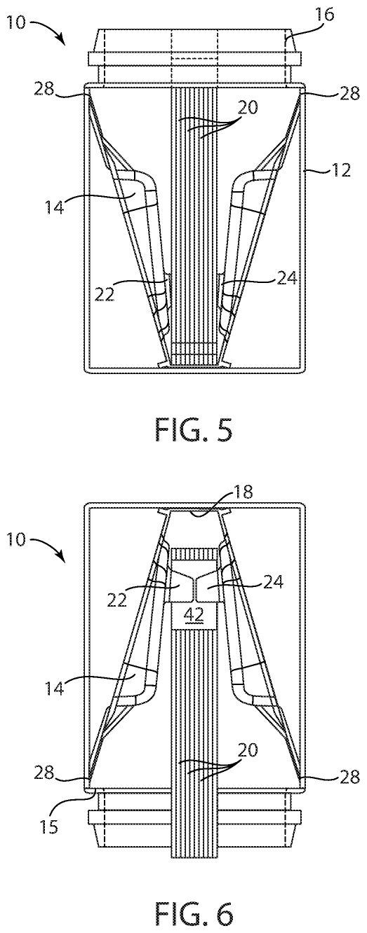

FIGS. 5 and 6 are additional cross-sectional views of a device 10 according to the disclosure, illustrating an additional inventive feature that facilitates dispensing of a pouch 20 and the thin strip medication contained therein. As noted above, aged or infirm individuals or those afflicted with neurological or muscular disorders often have impaired manual dexterity and the removal of a pouch from inside of a container can require some degree of fine motor skills in pinching an individual pouch and pulling it from its support (the rail formed by spars 22 and 24). As illustrated in FIG. 5, pouches 20 are designed to reside in container 20. When the container is upright (cap on top) the pouches either do not protrude beyond the rim at the mouth of the container or they are limited to extend only as far as the cap will permit.

However, when the cap is removed and the container is inverted as shown in FIG. 6, the pouches can protrude further out of the container because the mounting hole 42 of each pouch that joins it to the rail is larger than the thickness of the rail itself. Preferably the difference between the size of the pouch mounting hole and the thickness of the rail is at least 1 mm, preferably more than 2 mm. More generally, the difference in size can range from 0.5 mm to 2 cm, preferably between 2 mm and 5 mm so that the pouches can extend partially out of the opening of the container when the container is uncapped and inverted.

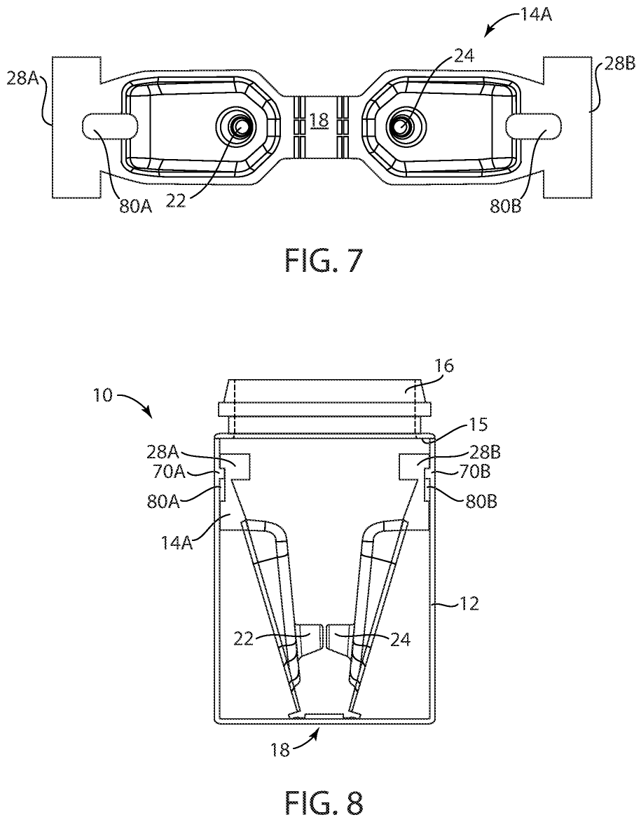

FIGS. 7 and 8 illustrate an alternative embodiment (or supplemental feature) of the disclosure. Device 10A in this embodiment again comprises a container 12 having an open mouth 13 and an inner chamber 17. Likewise, the device can comprises a child-resistant locking cap and a dispensing tray 14A. The tray 14A is again preferably a foldable tray configured to fold along a hinge region 18 for insertion through the mouth of the container 12. Once folded and inserted, end portions 28A and 28B can engage the perimeter of the container 12 to block removal.

In the embodiment of FIGS. 7 and 8, the end portions 28A, 28B can be laterally enlarged such that one or both of the end portions is capable of extending circumferentially along the container perimeter to secure the tray within the container 12. The end portions can be formed in a curved shape during manufacturing or they can be sufficiently compliant such that they will simply flex to extend circumferentially as a result of tray folding and deployment in the container 12. Preferably, at least one of the end portions 28A, 28B can circumferentially extend along the perimeter of the container to subtend an angle of at least 10 degrees, more preferably at least 20 degrees, or at least 30 degrees, or at least 40 degrees.

Another optional feature of tray 14A is a sliding engagement of the tray with the container. As shown in FIGS. 7 and 8, the tray 14a comprises slots 80A and 80B which can receive pin-like protrusions 70A and 70B on the inner surface of the container 12. The engagement of the pins 70A, 70B in their respective tray slots 80A, 80B prevents the tray from being dislodged once the pouches are loaded onto tray 14A and the tray is deployed within the container. (The pins can also prevent rotation of the tray within the container after deployment—to provide additional rigidity to the dispenser.) However, because the slots are elongated, some vertical movement is permitted. Thus, this sliding engagement can also permit the pouches (not shown in FIG. 8) to extend partially out of the opening of the container when the container is uncapped and inverted.

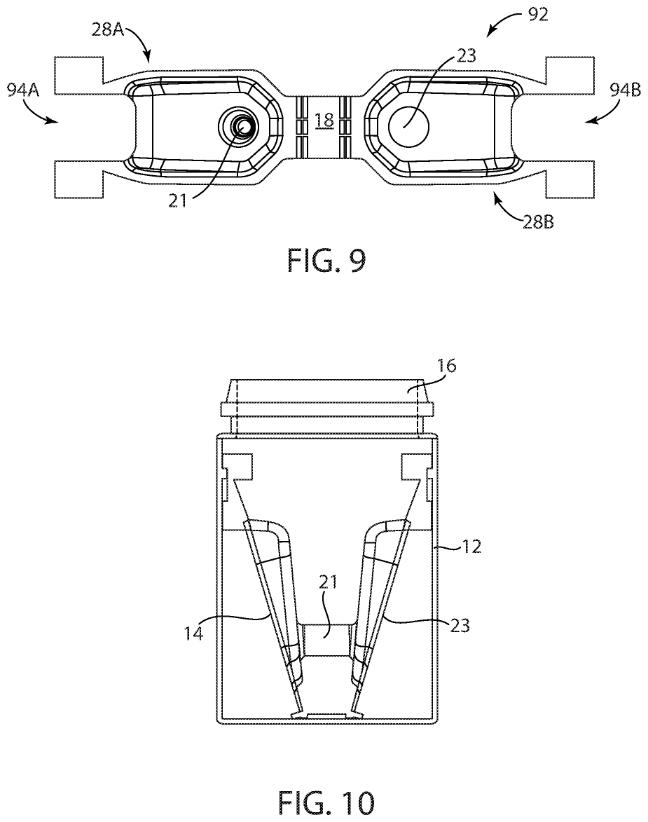

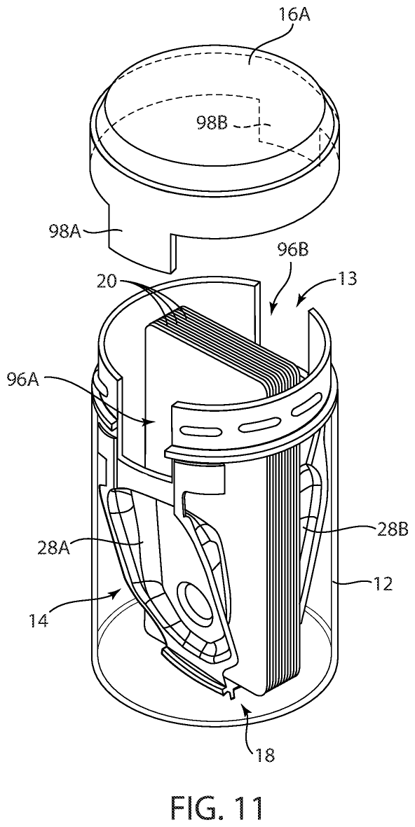

FIG. 9 is a side view of another alternative embodiment of a dispenser tray 92 according to the disclosure employing a single rail 21 for support of pouches. As in other embodiments the tray 92 comprises two arms 28A and 28B, foldable about hinge region 18. In the shown embodiment, Tray 92 is configured such that upon folding the single spar 21 can mate with a receptacle or mount 23. The connection between spar 21 and its mount 23 optionally can be a snap-fasten connection. Arms 28A and 28B also are shown with thumb-sized notches 94A and 94B, respectively, for reasons explained further in connection with FIG. 11.

FIG. 10 is a side view of a container 12 and cap 16 according to the disclosure with the tray 14 of FIG. 9 deployed therein and the spar 21 and mount 23 connected to form a support (rail) for pouches as discussed above in connection with other embodiments.

FIG. 11 is expanded perspective view of yet another embodiment of a device according to the disclosure having “thumb notches” 96A and 96B along the container mouth 13 to further assist a user in removing pouches. By notching the container 12 (and the tray 14 as shown in FIG. 9), a user can more easily reach into the container to remove a pouch 20. To seal the container 12, a modified cap 16A can be employed with flaps 98A and 98B configured to cover the container's thumb-notches when the cap is in a closed position. Again, cap 16A is preferably provides a child-resistant closure, e.g., a “quarter-turn,” “push-and-turn” or “squeeze-and-twist” mechanism.

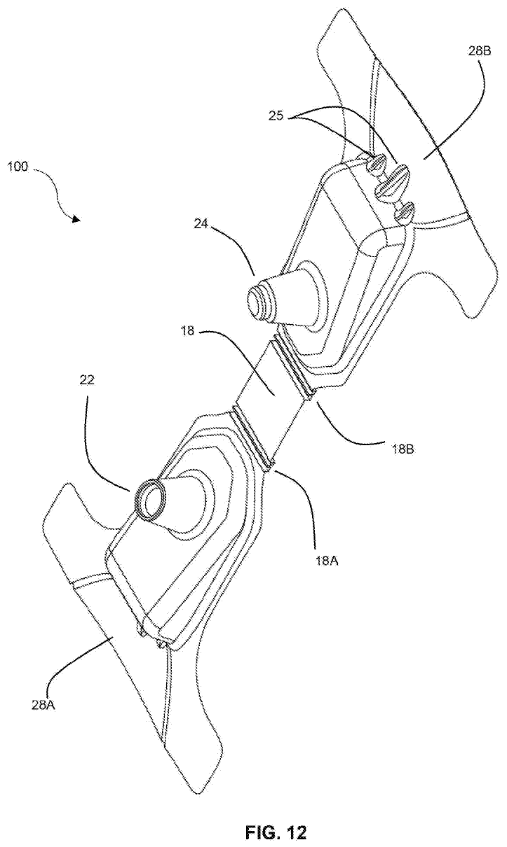

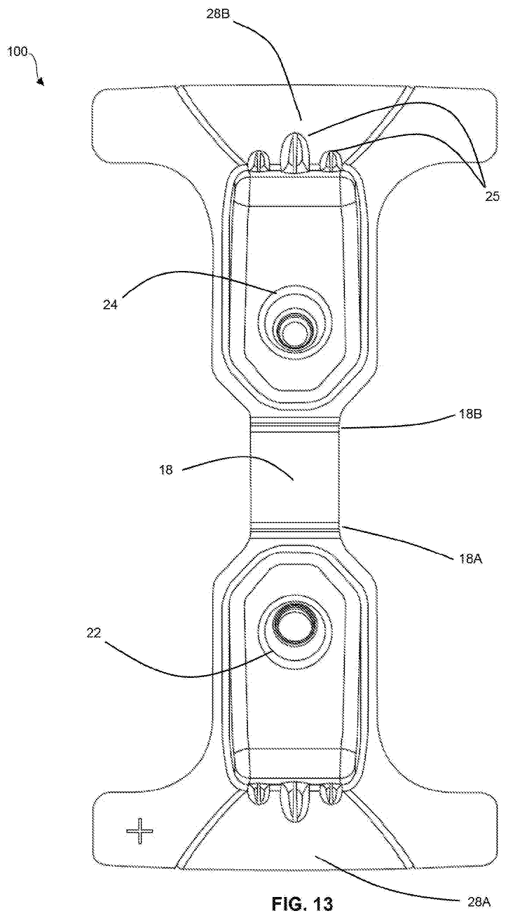

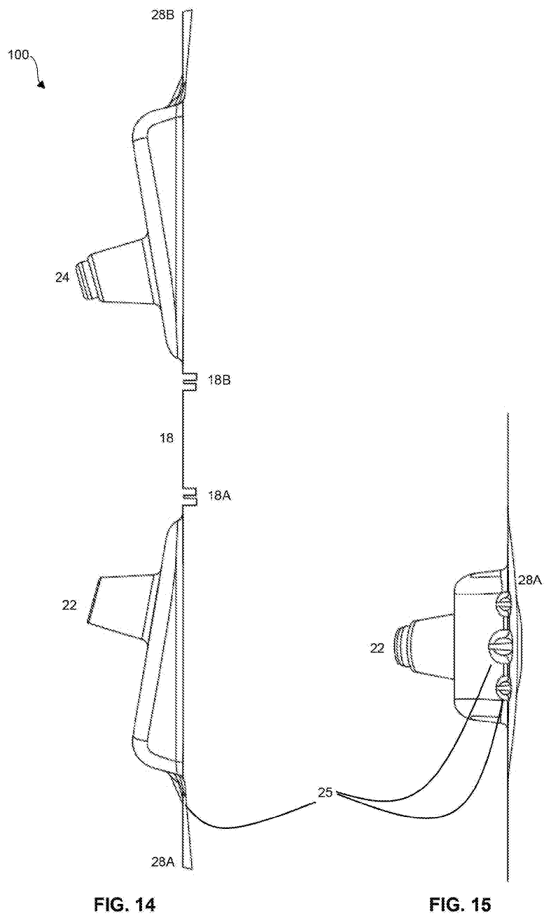

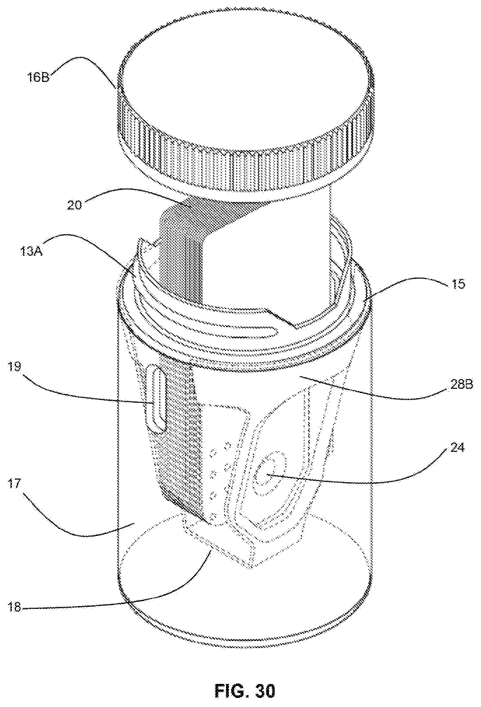

FIG. 12 is a perspective view of dispensing tray 100 according to the disclosure in its pre-folded configuration. FIG. 13 is a plan view and FIGS. 14 and 15 are side views of dispensing tray 100. The tray comprises hinge region 18 and arms 28A, 28B. Struts 25 provide additional structural rigidity to inhibit deformation except along the edges 18A and 18B of hinge region 18. Distal end portions of the arm 28A and 28B can be curved to further conform to the inner rim 15 around the mouth 13 of container 12 (e.g., FIG. 30). Spars 22 and 24 can be adapted to engage one another to form a rail when the tray is folded, e.g., via a male-female coupling or via any one of a variety of mating or snap-fastening type fittings.

FIG. 16 is a perspective view of empty container 110 that is configured to accommodate folded tray 100 or 130 and pouches 20. FIG. 17 is a side view, FIG. 18 is a top view, and FIG. 19 is a bottom view of empty container 110. Container 110 includes mouth 13 and inner chamber 17. Mouth 13 includes threads 13A for engaging complementary grooves or threads disposed along the inner circumference of cap 16B (e.g., FIG. 30). Mouth 13 may accommodate one or multiple threads for engaging complementary grooves or threads disposed along the inner circumference of cap 16B. Cap 16B may include, e.g., ribbed outer circumference to assist a user within removing cap 16B from container 110. Alternatively, cap 16B may include outer circumference without any tactile features (e.g., outer circumference of cap 16B may be smooth). Mouth 13 also includes thumb notches 96A and 96B. Thumb notches 96A and 96B can assist a user in removing pouches. Alternatively, mouth 13 may be included without thumb notches, e.g., in embodiments having a single thread disposed around the circumference of mouth 13. Container 110 includes inner rim 15. When folded tray 100 or 130 is placed in inner chamber 17, end portions of arms 28A and 28B engage inner rim 15 to block removal of tray 110. Inner chamber 17 includes two inward protrusions 19. These inward protrusions may limit or prevent rotation of the folded dispensing tray inside inner chamber 17.

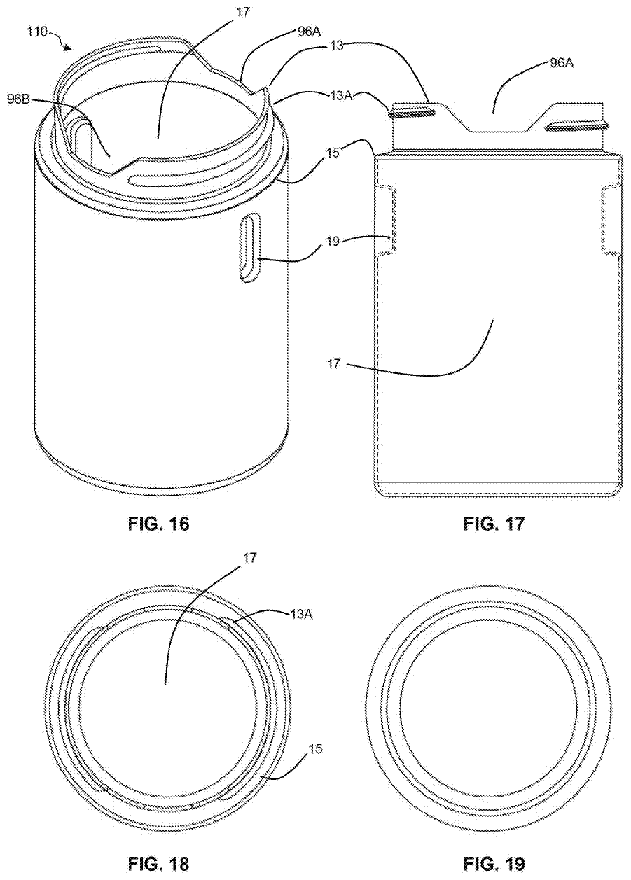

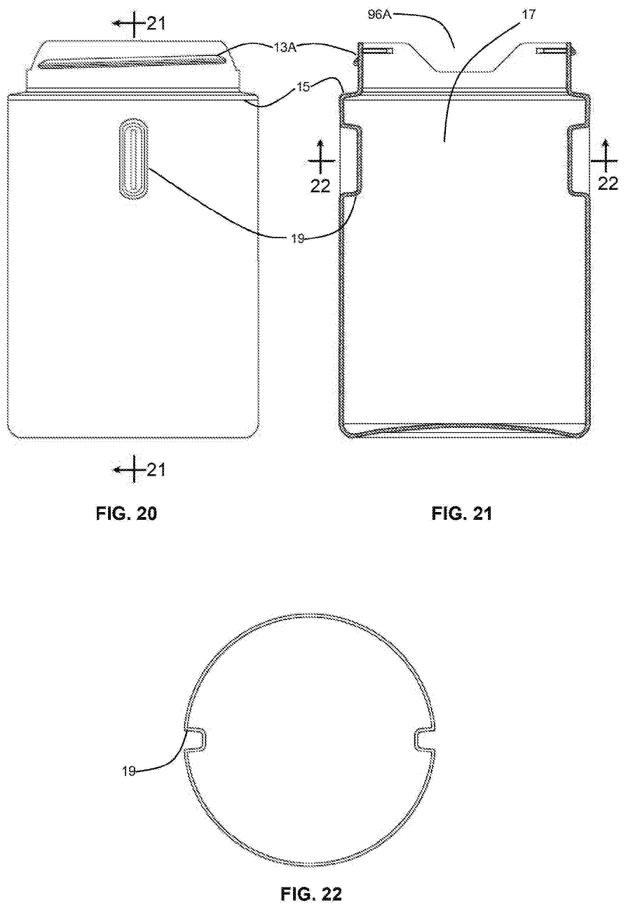

FIG. 20 is a side view of empty container 110 that is configured to accommodate folded tray 100 or 130 and pouches 20. FIG. 21 is a vertical cross-section view of container 110; the cross-section plane is indicated in FIG. 20 with normal vectors 21. FIG. 22 is a horizontal cross-section view of container 110; the cross-section plane is indicated in FIG. 21 with normal vectors 22.

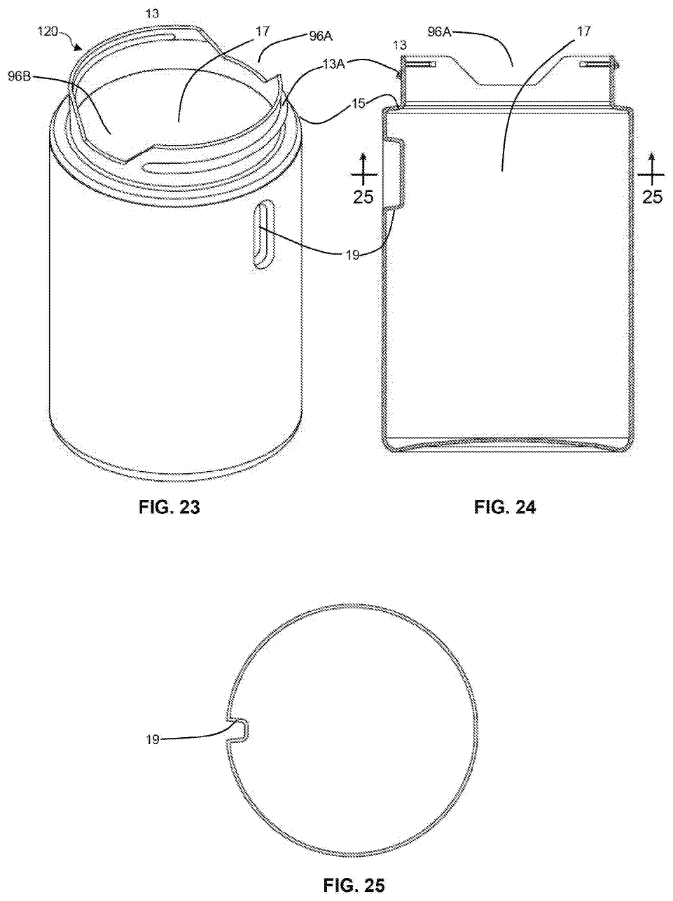

FIG. 23 is a perspective view of empty container 120 that is configured to accommodate folded tray 100 or 130 and pouches 20. FIG. 24 is a vertical cross-section view of container 110. FIG. 25 is a horizontal cross-section view of container 110; the cross-section plane is indicated in FIG. 24 with normal vectors 25. Container 120 is similar to container 110 and differs from the latter only in that inner chamber 17 of container 120 includes one inward protrusion 19. A single inward protrusion 19 is sufficient to limit or prevent rotation of the folded dispensing tray inside inner chamber 17. Devices described herein may have an inner chamber, e.g., with one, two, three, or four inner protrusions. In some instances, the inner chamber having four inner protrusions may be used, e.g., with a folded tray lacking wings.

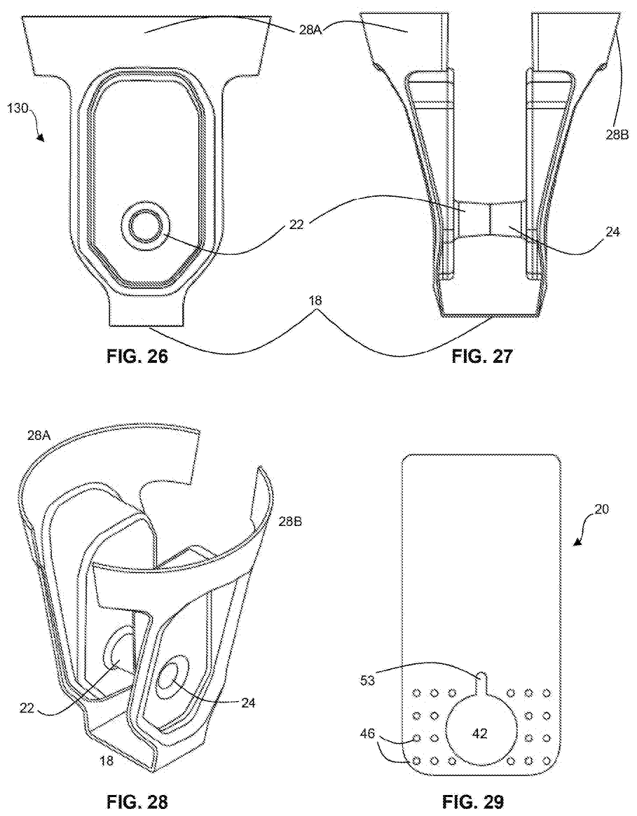

FIGS. 26 and 27 are side views of dispensing tray 130 in its folded configuration. FIG. 29 is a perspective view of dispensing tray 130 in its folded configuration. Dispensing tray 130 is similar to dispensing tray 110 and differs from the latter in its lacking struts 25.

FIG. 29 is a plan view of pouch 20.

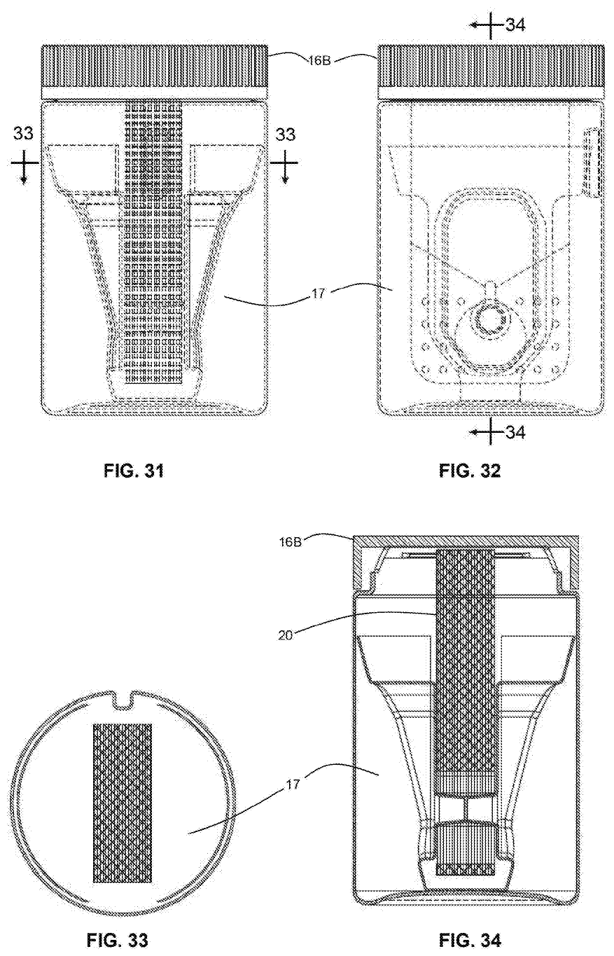

FIG. 30 is an expanded, perspective view of container 110, dispensing tray 130, pouches 20, and cap 16B. FIGS. 31 and 32 are side views of a dispenser including container 110, dispensing tray 130, pouches 20, and cap 16B assembled together. FIG. 33 is a horizontal cross-section view of the dispenser; the cross-section plane is indicated in FIG. 31 with normal vectors 33. FIG. 34 is a vertical cross-section view of the dispenser; the cross-section plane is indicated in FIG. 32 with normal vectors 34.

The embodiments of the present technology described herein are not intended to be exhaustive or to limit the technology to the precise forms disclosed in the following detailed description. Rather, the embodiments are chosen and described so that others skilled in the art can appreciate and understand the principles and practices of the present technology. The technology has been described with reference to various specific and preferred embodiments and techniques. However, it should be understood that many variations and modifications may be made while remaining within the spirit and scope of the technology. Unless technically impossible, any feature or element described in connection with one embodiment can be interchangeably used, or additively combined, with any of the features or elements of each and every other embodiment and all such permeations are encompassed by the present disclosure.

All publications, patents, and patent applications in this specification are indicative of the level of ordinary skill in the art to which this technology pertains. All publications, patents, and patent applications are herein incorporated by reference to the same extent as if each individual publication or patent application was specifically and individually indicated by reference.

What is claimed is:

1. A device comprising:
a container having an opening and an inner chamber,
a cap configured to close the opening of the container, and
a tray deployable in the chamber of the container and configured to hold at least one pouch carrying a product.

2. The device of claim 1, wherein the device is a medication dispenser, the cap is a child-resistant cap, and the tray deployable in the chamber of the container is configured to hold at least one pouch carrying a dose of medication.

3. The device of claim 1 or 2, wherein the tray comprises a support configured to hold one or more pouches when the tray and pouches are deployed within the container, and optionally wherein the support comprises a rail onto which the pouches can be individually coupled to facilitate dispensation of one pouch at a time by pulling the pouch from the rail.

4. The device of claim 3, wherein each of the pouches comprises an opening for attachment to the rail.

5. The device of claim 4, wherein the openings of the pouches are larger than the thickness of the rail such that the pouches can extend partially out of the opening of the container when the container is uncapped and inverted.

6. The device of any one of claims 1 to 5, wherein the tray is foldable to facilitate insertion of the tray into the chamber of the container.

7. The device of any one of claims 1 to 6, wherein the tray comprises at least two arms and a hinge about which the arms can fold.

8. The device of claim 7, wherein at least one arm of the tray further comprises a spar that forms a rail to support the pouch when the tray is folded, and optionally wherein the spar couples to the second arm when the tray is folded.

9. The device of any one of claims 1 to 7, wherein the tray further comprises a first spar and a second spar that engage with each other to form the rail when the tray is deployed within the chamber of the container.

10. The device of claim 9, wherein one of the spars has a male end and the other has a female end that can be joined together to form the rail, and optionally wherein the first and second spars are configured to snap-fasten together to form the rail.

11. The device of claim 7, wherein each arm further comprises a flange configured to engage an inner rim of the container to impede removal of the tray after insertion into the chamber of the container.

12. The device of claim 11, wherein the flange of at least one arm is shaped to extend circumferentially along the container rim such that it subtends an angle of at least 10 degrees.

13. The device of claim 11 or 12, wherein the flange of each arm is shaped to extend circumferentially along the container rim such that it subtends an angle of at least 30 degrees.

14. The device of any one of claims 1 to 13, wherein the container further comprises at least one post that protrudes inwardly into the chamber of the container to engage the tray when deployed therein, and the tray comprises an opening for receiving the post.

15. The device of claim 14, wherein the tray opening is a longitudinal slot that permits limited longitudinal movement of the tray within the chamber such that the tray and associated pouches can extend partially out of the opening of the container when the container is uncapped and inverted.

16. The device of any one of claims 1 to 15, wherein the cap and container cooperate to provide a child resistant locking engagement.

17. The device of any one of claims 1 to 16, wherein the engagement of the cap and container can be unlocked by a quarter turn action, or push and twist action by a squeeze and twist action.

18. The device of any one of claims 1 to 17, further comprising a plurality of pouches.

19. The device of claim 18, wherein each pouch contains a dose of a thin strip medication.

20. The device of claim 19, wherein the thin strip medication is an orally-dissolvable medication.

21. The device of any one of claims 1 to 20, wherein the inner chamber comprises at least one inner protrusion.

22. The device of any one of claims 1 to 21, wherein the cap comprises a plurality of vertical ribs disposed along the outer circumference of the cap.

23. The device of any one of claims 1 to 22, wherein the cap comprises a mouth having one or more threads disposed along its circumference.

24. A method of dispensing a product comprising:
providing at least one sealed pouch containing a product,
folding a tray around the pouch such that the tray supports the pouch,
placing the tray and supported pouch inside a container such that the tray is engaged by the container in a manner that impedes removal of the tray, and
securing the container with a cap configured to close the opening of the container,
whereby a user can remove the cap and obtain an individual pouch by reaching into the container and pulling the pouch from its supporting tray.

25. The method of claim 24, where the method further comprises a method of dispensing a medication, the pouch contains a dose of medication and the cap is a child-resistant cap.

26. The method of claim 24 or 25, wherein the tray comprises a support configured to hold a plurality of pouches when the tray and pouches are deployed within the container, and optionally the tray comprises a rail onto which the pouches can be individually coupled to facilitate dispensation of one pouch at a time by pulling the pouch from the rail.

27. The method of claim 26, wherein each of the pouches comprises an opening for attachment to the rail.

28. The method of claim 27, wherein the openings of the pouches are larger than the thickness of the rail such that the pouches can extend partially out of the opening of the container when the container is uncapped and inverted.

29. The method of any one of claims 24 to 28, wherein the tray is foldable along a hinge region to facilitate insertion of the tray into the chamber of the container.

30. The method of claim 29, wherein the tray comprises at least two arms and a hinge about which the arms can fold, and the step of placing the tray inside the container further comprises folding the arms about the hinge region and placing the hinge region in proximity to a bottom surface of the container.

31. The method of claim 30, wherein at least one arm of the tray further comprises a spar and the step of folding the tray further comprises forming a rail a rail to support the pouch when the tray is folded, and optionally wherein the spar couples to the second arm when the tray is folded.

32. The method of claim 30, wherein the tray further comprises a first spar on a first arm and a second spar on a second arm that engage with each other to form the rail when the tray is deployed within the chamber of the container, and the step of folding the tray further comprises forming the rail such that it passes through a hole in the pouch.

33. The method of claim 32, wherein one of the spars has a male end and the other has a female end that can be joined together to form the rail, and optionally wherein the first and second spars are configured to snap-fasten together to form the rail.

34. The method of claim 33, wherein each arm further comprises a flange configured to engage an inner rim of the container to impede removal of the tray after insertion into the chamber of the container.

35. The method of claim 34, wherein the flange of at least one arm is shaped to extend circumferentially along the container rim such that it subtends an angle of at least 10 degrees.

36. The method of claim 34 or 35, wherein the flange of each arm is shaped to extend circumferentially along the container rim such that it subtends an angle of at least 30 degrees.

37. The method of any one of claims 24 to 36, wherein the container further comprises at least one post that protrudes inwardly into the chamber of the container to engage at least one opening in the tray when deployed therein, and the step of placing the tray inside the container further comprises placing the tray such that the tray opening receives the post.

38. The method of any one of claims 24 to 37, wherein the tray opening is a longitudinal slot that permits limited longitudinal movement of the tray within the chamber such that the tray and associated pouches can extend partially out of the opening of the container when the container is uncapped and inverted.

39. The method of any one of claims 24 to 38, wherein the engagement of the cap and container can be unlocked by a push and twist action.

40. The method of any one of claims 24 to 38, wherein the engagement of the cap and container can be unlocked by a squeeze and twist action.

41. The method of any one of claims 26 to 40, wherein the inner chamber comprises at least one inner protrusion.

42. The method of any one of claims 24 to 41, wherein the cap comprises a plurality of vertical ribs disposed along the outer circumference of the cap.

43. The method of any one of claims 24 to 42, wherein the cap comprises a mouth having one or more threads disposed along its circumference.

44. The method of any one of claims 24 to 43, further comprising supporting a plurality of pouches with the tray inside the container.

45. The method of claim 44, wherein each pouch contains a dose of thin strip medication.

46. The method of claim 45, wherein thin strip medication is orally-dissolvable medication.

47. A method of treating a subject afflicted with a neurological condition comprising:
providing at least one sealed pouch containing a medication,
folding a tray around the pouch such that the tray supports the pouch,
placing the tray and supported pouch inside a container such that the tray is engaged by the container in a manner that impedes removal of the tray, and
securing the container with a cap configured to close the opening of the container,
thereby allowing the subject to remove the cap and obtain an individual pouch by reaching into the container and pulling the pouch from its supporting tray and further permitting the subject to open the pouch and administer to themselves the medication.

48. The method of claim 47, wherein the cap is a child-resistant cap and the method further comprises releasing the interlocking engagement of the cap and container.

49. The method of claim 47 or 48, wherein the neurological condition is Parkinson's Disease.

50. The method of any one of claims 47 to 49, wherein the neurological condition is an off episode associated with Parkinson's Disease.

51. The method of any one of claims 47 to 50, wherein the medication comprises a thin strip medication deployed within the pouch.

52. The method of any one of claims 47 to 51, wherein the method further comprises opening the pouch by pulling on opposing tabs of the pouch to separate the pouch into at least two sheet elements and allow the subject to access the thin strip medication deployed between the sheet elements.

53. The method of any one of claims 47 to 52, wherein the medication comprises apomorphine or a pharmaceutically acceptable salt thereof.

54. The method of claim 53, wherein the apomorphine or a pharmaceutically acceptable salt thereof is formulated for sublingual release and the method permits the subject to place the thin strip medications under their tongue.
